(19) United States
(12) Patent Application Publication
(10) Pub. No.: US 2011/0195474 A1
(43) Pub. Date: Aug. 11, 2011

(54) METHODS OF USING A MULTIPLE SHEATH FLOW DEVICE FOR THE PRODUCTION OF MICROCAPSULES

(75) Inventor: Kevin Ulmer, Woods Hole, MA (US)

(73) Assignee: Genome Corporation, Providence, RI (US)

(21) Appl. No.: 13/057,976

(22) PCT Filed: Aug. 6, 2009
(86) PCT No.: PCT/US09/52933

§ 371 Date: Apr. 28, 2011

Related U.S. Application Data
(63) Continuation-in-part of application No. 12/221,792, filed on Aug. 6, 2008.

(51) Int. Cl. C12N 11/00; B29C 39/12

(52) U.S. Cl. 435/174; 264/4

(57) ABSTRACT

The present invention provides methods for preparing micro-capsules, each microcapsule comprising a semipermeable membrane, an aqueous core, one or more enzymes in the aqueous core, and one nucleic acid template in the aqueous core. Microcapsules are prepared by flowing (i) an innermost fluid flow comprising at least one enzyme and at least one nucleic acid template, (ii) a middle fluid flow comprising a semipermeable membrane forming material, and (iii) an outer fluid flow comprising a solution or gas through a multiple sheath flow device. The outer fluid flow entrains the innermost fluid flow and middle fluid flow in the aperture of the multiple sheath flow device. The fluids exit the multiple sheath flow device as a liquid jet resulting in microcapsules.

### RELATED APPLICATION

This application is related to and claims the benefit of U.S. nonprovisional patent application Ser. No. 12/221,792, filed Aug. 6, 2008, the contents of which are incorporated herein by reference in their entirety.

### FIELD OF INVENTION

This application relates to a multiple sheath flow device and methods of using same for the production of microcapsules. The microcapsules produced by the methods of the invention can be used in enzyme-mediated reactions, specifically, nucleic acid amplification and nucleic acid sequencing.

### BACKGROUND OF INVENTION

DNA sequencing encompasses biochemical methods for determining the order of nucleotide bases (adenine, guanine, cytosine and thymine) in a nucleic acid. Sanger sequencing, otherwise known as dideoxynucleotide sequencing or chain-termination sequencing, was developed in the mid-1970's and continues to be widely used in DNA sequencing. The Sanger sequencing method utilizes the incorporation of 2′, 3′-dideoxynucleotide triphosphates (ddNTPs) in a growing DNA chain. To perform a Sanger sequencing reaction, a single stranded DNA template, a primer, polymerase, deoxynucleotide triphosphates (dNTPs) and one or more ddNTPs are mixed together and incubated under conditions that allow for primers to anneal to the single stranded DNA and elongate to form a complementary DNA strand. Either dNTPs, primer or ddNTPs are labeled in the reaction. During elongation, both dNTPs and ddNTPs (usually at a concentration of about 1% total concentration of dNTPs) are incorporated in the complementary DNA strand. Unlike dNTPs, ddNTPs lack a 3′-OH group and are unable to form a phosphodiester bond with the next deoxynucleotide. As a result, the DNA chain is terminated at the point of incorporation of the ddNTP. The sequence of the DNA is determined by separating the resulting labeled DNA fragments by electrophoresis. See, Sanger, F. and Coulson, A. R., 1975, J. Molec. Biol. 94: 441-448; Sanger, F. et al., 1977, Nature. 265(5596): 687-695; and Sanger, F. et al., 1977, Proc. Natl. Acad. Sci. U.S.A. 75: 5463-5467.

Despite being a commonly-used method, Sanger sequencing is expensive, labor intensive and not well suited for high-throughput sequencing. As a result, several amplification and sequencing technologies have been developed in attempts to overcome the drawbacks associated with Sanger sequencing. Amplification technologies developed to be used in conjunction with sequencing include, for instance, emulsion PCR and bridge PCR. New sequencing technologies include, for instance, pyrophosphate sequencing, base-at-a-time sequencing by synthesis, and supported oligo ligation detection. However, the read lengths produced by many of these newer technologies tend to be shorter than those produced by Sanger sequencing and the raw accuracy of the reads tends to be lower.

A major problem that has faced researchers has been the inability to prepare in a short period of time and at low cost the numerous nucleic acid samples necessary for large scale sequencing. Sanger sequencing as well as many other sequencing methods require amplified nucleic acid template as starting material. Methods typically used for amplifying nucleic acid template for subsequent sequencing such as cloning and random-primed PCR are ill-suited for large scale sequencing. Cloning is labor intensive and, as a result, both time consuming and costly. Cloning biases can also occur which may prevent some nucleic acid regions from being represented. Although random-primed PCR is not as laborious as cloning and can be engineered to amplify a plurality of nucleic acids in a single reaction, the method is not preferred because the amplified nucleic acids are usually not representative of the starting nucleic acid templates (i.e., amplification bias occurs).

Emulsion PCR is an example of an amplification method that has been developed to amplify nucleic acids for large scale sequencing. In emulsion PCR, a nucleic acid template, primer coated bead, and amplification solution are suspended in a heat stable water-in-oil emulsion to form thousands to millions of separate microreactors. Each microreactor optimally contains a single nucleic acid template and a single bead. Within each microreactor, nucleic acid amplification takes place on the bead and amplified nucleic acid fragments remain attached to the bead. By placing thousands to millions of microreactors in a single tube and subjecting the tube to conditions necessary for PCR, thousands to millions of nucleic acid templates can be amplified in parallel without competition among the reactions. Sequencing of amplified nucleic acid fragments can subsequently be performed by breaking the microreactors and isolating the beads containing amplified nucleic acid fragments. See, for instance, U.S. Patent Application 200500079510; Margulies, M. et al., 2005, Nature. 437: 376-380; Shendure, J. et al., 2005,Science. 309: 1728-1732; Williams, R. et al., 2006, Nature Methods. 3(7): 545-550; and Wicker, T. et al., 2006,BMC Genomics. 7: 275.

Sanger sequencing relies on electrophoresis methods to “read” a nucleic acid sequence after a sequencing reaction has been performed. Electrophoresis methods separate nucleic acid fragments based on size so that fragments differing by only one nucleotide can be resolved. Most genomic sequencing strategies to date have relied on capillary array electrophoresis (CAE). For instance, CAE was used by the Celera human sequencing project to sequence cloned nucleic acid fragments, which were then assembled electronically (Fredlake, C. et al., 2006, Electrophoresis. 27: 3689-3702). Commercial CAE instruments are able to produce raw sequencing reads of about 650-700 phred 20 (Q20) quality bases per capillary in about 1-2 hours with 100-400 capillaries per instrument. Id.

Other electrophoretic methods and non-electrophoretic methods have been developed for use with sequencing, including, but not limited to, microfluidic devices such as on-chip sequencing systems and massively parallel sequencing systems for use with sequencing by synthesis methods. Reports of read lengths produced by microfluidic devices varies greatly from about 320 bases to over 800 bases depending on sequencing system used. Massively parallel sequencing systems, for instance, 454 Life Sciences' GS-20, which uses an enzymatic sequencing by synthesis technique known as pyrosequencing, reportedly averages a read length of 100 bases and can read up to 25 million bases in one 4-hour run. Id. Although the GS-40 seems well suited for sequencing small genomes such as bacterial and viral genomes, the shorter read lengths obtained with this system relative to Sanger sequencing make this system less suitable for sequencing larger genomes at present.

### SUMMARY OF THE INVENTION

The present invention provides materials and methods that enable massively-parallel sample preparation and nucleic acids sequencing in a single reaction vessel. The invention allows single-vessel, batch processing of samples to produce sequence for high-depth coverage of large genomes. Using compartmentalized sequencing vesicles as taught herein, a user is able to conduct single molecule amplification and Sanger sequencing reactions in the same compartment with free exchange of reagents and by-products, but not large macromolecules such as nucleic acids and polymerase.

Thus, according to the invention, sample preparation and sequencing are conducted in a semi-closed environment in which one can produce a clonal population of the nucleic acids to be sequenced. In a preferred embodiment, the vesicles are semi-permeable membrane droplets housing an aqueous compartment for conducting DNA amplification and sequencing reactions. Use of the invention allows one to conduct massively-parallel sequencing reactions, especially Sanger-type reactions, in a self-contained environment.

The present invention also provides methods of using a multiple sheath flow device for the production of microcapsules, each microcapsule comprising a semipermeable membrane, an aqueous core, one or more enzymes in the aqueous core, and a nucleic acid template in the aqueous core for use in an enzyme-mediated reaction. The invention includes methods for the production of microcapsules, wherein each microcapsule comprises a semipermeable membrane surrounding one or more polymerase enzymes and a nucleic acid template. The microcapsules produced by methods of the invention are used to amplify a nucleic acid template by rolling circle amplification (RCA) or polymerase chain reaction (PCR) and subsequently sequence the amplified nucleic acid template. Further, microcapsules produced by the methods of the invention are used to sequence an amplified nucleic acid template using Sanger sequencing biochemistry but on a larger scale and without the labor-intensive sample preparation steps generally associated with Sanger sequencing.

A plurality of microcapsules can be used to amplify thousands, even millions or hundreds of millions, of nucleic acid templates simultaneously in a single reaction vessel. Although emulsion PCR can also be used to amplify thousands of nucleic acids in a single reaction, emulsion PCR is limited by the impermeable nature of the oil surrounding the aqueous droplets. Unlike an oil emulsion, the semipermeable membrane of the microcapsules produced by the methods of the present invention allows for the free exchange of low molecular weight molecules and reagents (e.g., dNTPs, fluorescently labeled ddNTPs, short primers) and reaction byproducts (e.g., pyrophosphate). As a result, the microcapsules provide a near constant concentration of reaction reagents and substrates with the ability to remove inhibitory byproducts by diffusion. This property when coupled with Sanger sequencing biochemistry allows for the amplification and subsequent sequencing of longer nucleic acid templates than is possible with current emulsion PCR methods. For instance, the microcapsules produced by the methods of the present invention can be used to produce reads of about 800 to 1,000 high quality bases, whereas the 454 Life Sciences sequencing technique which uses emulsion PCR in conjunction with pyrosequencing is reported to only produce reads of about 100 to 200 high quality bases (Wicker, T. et al., 2006, BMC Genomics. 7: 275).

### BRIEF DESCRIPTION OF THE DRAWINGS

FIG. 1 is a diagram of an exemplary multiple sheath flow device for use in making microcapsules according to the present invention.

FIG. 2 is an expanded view of the device shown in FIG. 1.

FIG. 3 provides dimensions of the device shown in FIG. 1.

FIG. 4 depicts the flow paths of the device shown in FIG. 1.

FIGS. 5A and 5B are bright field and fluorescent images, respectively, of impermeable polymer shell microcapsules 6 days post-encapsulation.

FIG. 6 is a bright field image of smaller diameter impermeable polymer shell microcapsules.

FIGS. 7A-L are bright field and fluorescent images of intermediate diameter and/or thinner impermeable polymer shell microcapsules.

FIGS. 8A-D are bright field and fluorescent images of permeable polymer shell microcapsules at 5 minutes and 20 hours post-encapsulation.

FIGS. 9A-T are bright field and fluorescent images of semi-permeable polymer shell microcapsules at 5 minutes and 16 hours post encapsulation.

FIGS. 10A-F are bright field and fluorescent images of higher molecular weight cut-off semi-permeable polymer shell microcapsules.

FIG. 11A-H are bright field and fluorescent images of semi-permeable polymer shell microcapsules containing DNA.

FIG. 12A-L are bright field and fluorescent images of semi-permeable polymer shell microcapsules used for Rolling Circle Amplification of encapsulated DNA.

FIG. 13A-L are bright field and fluorescent images of Rolling Circle Amplification in alternative formulation semi-permeable polymer shell microcapsules.

FIG. 14A-D are bright field and fluorescent images demonstrating thermostability of semi-permeable polymer shell microcapsules.

FIG. 15A-F are bright field and fluorescent images demonstrating permeability of polymer shell microcapsules to dye-labeled dideoxyterminators.

### DETAILED DESCRIPTION

The present invention includes methods of making microcapsules. In one embodiment of the invention, the multiple sheath flow device is used to produce microcapsules, each microcapsule comprising a semipermeable membrane, aqueous core, one or more enzymes in the aqueous core, and a nucleic acid template in the aqueous core. In another embodiment of the invention, each microcapsule produced by the methods of the invention comprises a semipermeable membrane, an aqueous core, one or more enzymes in the aqueous core, and a nucleic acid template in the aqueous core.

### Multiple Sheath Flow Device

A multiple sheath flow device of the present invention comprises at least three inlet connections which serve as portals of entry for (a) a liquid comprising nucleic acid templates and one or more enzymes which are to be incorporated in the aqueous core of the microcapsules, (b) a semipermeable membrane forming material, and (c) a third solution or gas. The three fluids enter the multiple sheath flow device through tubing wherein the liquid flow containing nucleic acid templates and one or more enzymes forms an innermost liquid flow, the semipermeable membrane forming material forms a middle coaxial flow, and the third solution or gas forms an outer liquid flow. The multiple sheath flow device further comprises an aperture through which the combined coaxial flows are focused. In the aperture, the solution or gas that forms the outer liquid flow entrains the innermost and middle liquid flows. The fluids emerge through the aperture as a liquid jet. Breakup of the jet into microcapsules occurs beyond the aperture, either passively, as determined by (1) the geometry of the flow focusing device, (2) the rheological properties of the fluids, and (3) their relative flow rates, or actively by an externally applied oscillatory field (e.g., from an acoustic transducer or piezoelectric crystal). Other multiple coaxial sheath flow devices are known in the art that operate in a flow regime known as dripping mode rather than jetting mode.

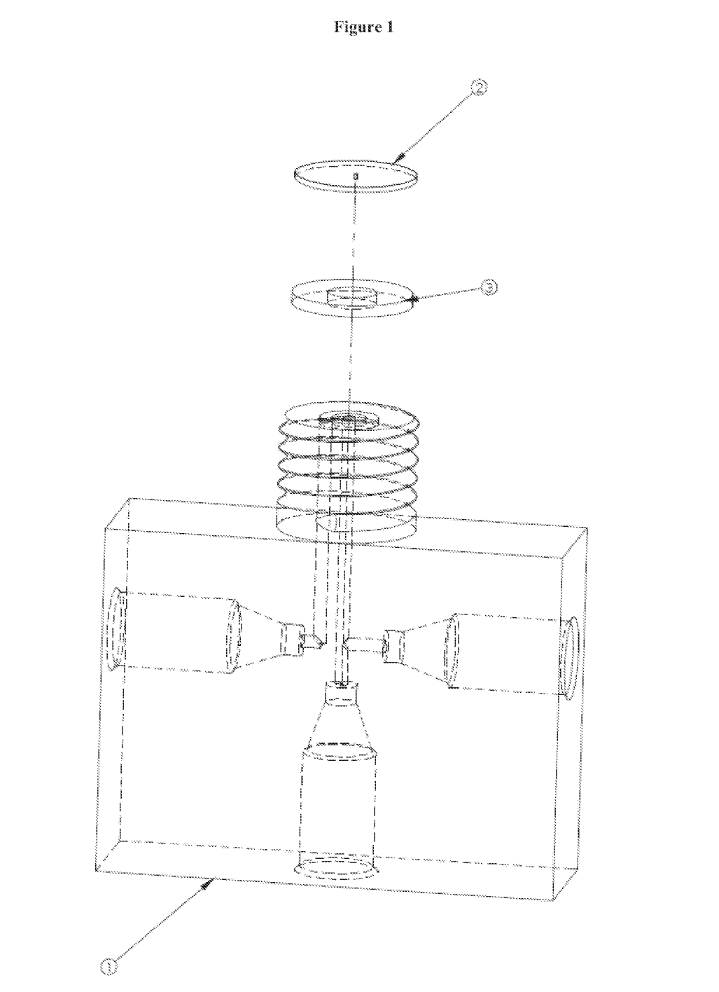

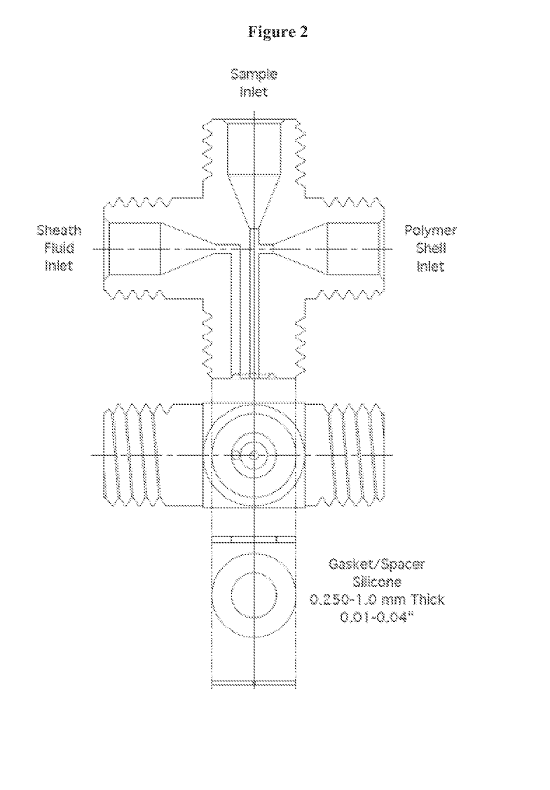

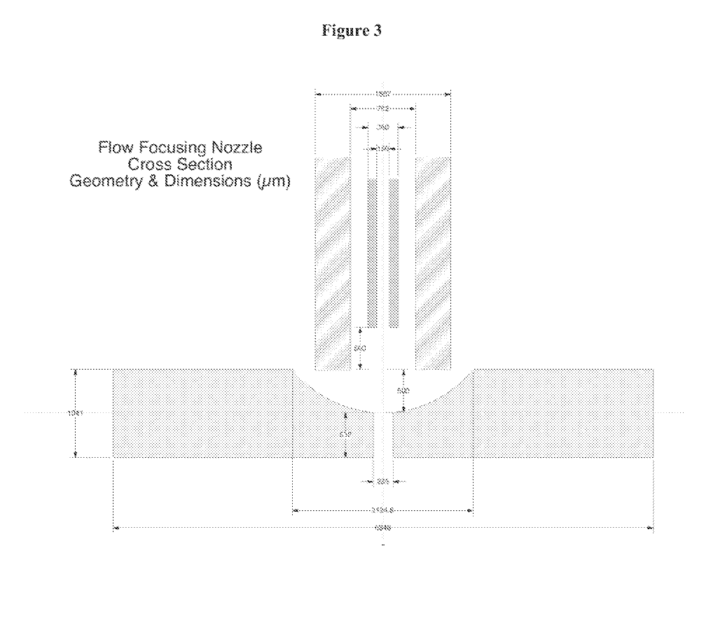

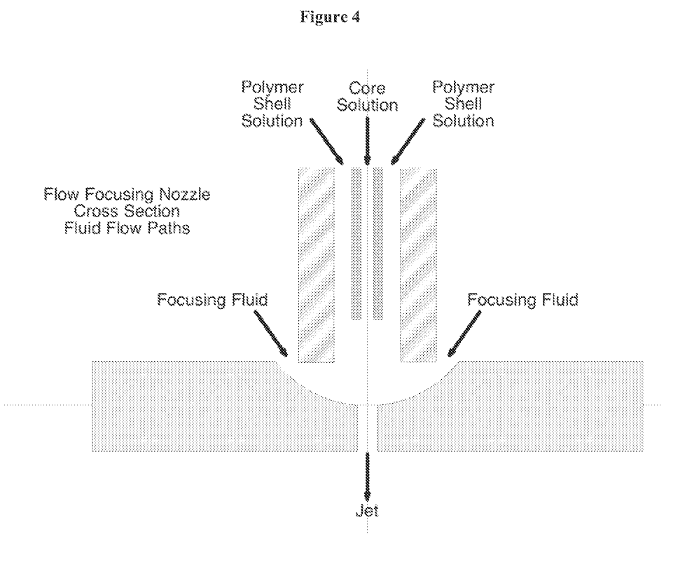

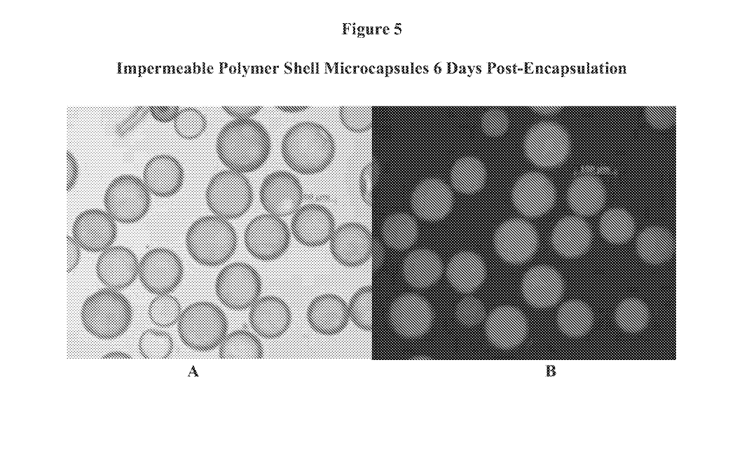

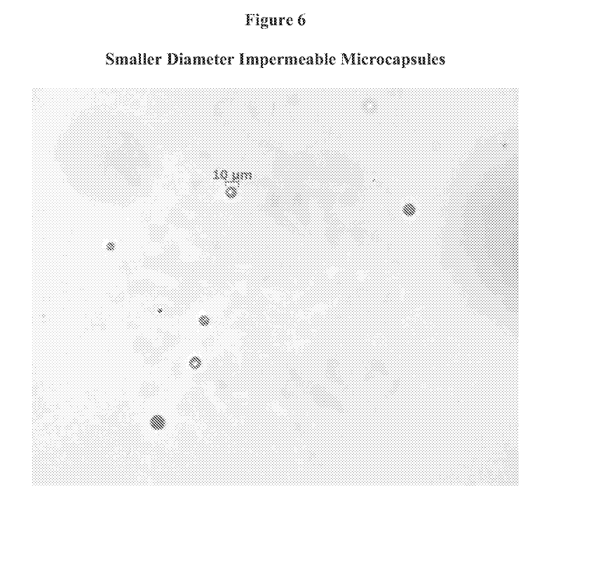

Diagrams of a multiple sheath flow device is provided in FIGS. 1-4. In FIG. 1, reference numeral 1 refers to the microdrop generator block that comprises inlets and flow channels for droplet formation. Reference numeral 2 shows a pinhole washer and reference numeral 3 shows a gasket/spacer for attachment of an outlet to the block. FIG. 2 shows an expanded view of the three inlets of a preferred embodiment of the invention, showing inlet ports for components of droplets for processing as described herein. FIG. 3 provides dimensions and geometry of the flow focusing nozzle, and FIG. 4 depicts the flow paths of the three fluids through the flow focusing nozzle.

A preferred device is machined from PEEK or a similar material and has three capillary tubing inlet connections (Upchurch): (a) the innermost liquid flow contains the diluted DNA fragments+DNA polymerase to be incorporated into the microcapsule, (b) a coaxial flow of immiscible semipermeable membrane forming material surrounds the inner core, and (c) an outer coaxial flow of a third solution or gas is used to entrain the microcapsules. The three coaxial fluids emerge through a precision sapphire aperture as a liquid jet. By adjusting the relative flow rates of the three fluid feeds, it is possible to independently control the diameter of the microcapsules that are formed (by Rayleigh instability of the jet or imposed by high-frequency oscillations of an attached piezo element) over a broad range (μm to nm) and the thickness of the encapsulating semipermeable membrane. The device can be operated at high pressure, using three computer-controlled syringe pumps, to increase the rate of microcapsule formation and the microcapillary design minimizes regent consumption. Multiple aperture designs allow parallel jet formation with even higher throughput. These microcapsules have a very uniform size distribution (i.e., they are essentially monodisperse). There is a wide range of materials suitable for forming the semipermeable membrane surrounding the microcapsule that can be chosen to control the molecular weight cutoff. Such materials include photocrosslinkable polymers and other polymers whose crosslinking can be controlled by the chemistry of the outermost liquid sheath.

Other configurations of devices of the invention are contemplated, consistent with the present disclosure. For example, two or more of the inlets can be combined (e.g., producing a device having two inlets). Alternatively, additional inlet connections can be made at the convenience of the manufacturer and user.

In one embodiment of the invention, inlet connections are inert capillary tubing inlet connections. In one embodiment of the invention, inlet connections are Upchurch inert capillary tubing inlet connections. In one embodiment of the invention, the aperture is a precision sapphire aperture (e.g., Bird Precision, Waltham, Mass.) or metal foil pinhole (Edmund Optics, Barrington, N.J.).

One or more multiple sheath flow devices of the invention may be combined in parallel for increased throughput.

Methods of Making Microcapsules Using a Multiple Sheath Flow Device

The present invention includes methods of making a microcapsule or a plurality of microcapsules using a multiple sheath flow device as described herein. The invention includes a method of making microcapsules for use in an enzyme-mediated reaction comprising (a) receiving an innermost liquid flow, a middle fluid flow and an outer fluid flow in a multiple sheath flow device through separate inlet connections, wherein the innermost liquid flow comprises one or more nucleic acid templates and one or more enzymes, the middle fluid flow comprises a semipermeable membrane forming material, and the outer fluid flow comprises a solution or gas and (b) combining the innermost and middle fluid flows in an aperture of the multiple sheath flow device.

In another method of the invention, microcapsules for use in a polymerase-mediated reaction are prepared comprising (a) receiving an innermost liquid flow, a middle fluid flow and an outer fluid flow in a multiple sheath flow device through separate inlet connections, wherein the innermost liquid flow comprises one or more nucleic acid templates and one or more polymerases, the middle fluid flow comprises a semipermeable membrane forming material, and the outer fluid flow comprises a solution or gas and (b) combining the innermost and middle fluid flows in an aperture of the multiple sheath flow device.

In the aperture of the multiple sheath device, the outermost fluid entrains the innermost and middle fluid flows. The fluids exit the aperture in a liquid jet. Microcapsules are formed by the liquid jet breaking up. Microcapsules can be subsequently placed in one or more tubes for use in a enzymatic reaction. Harvesting of the microcapsules for subsequent processing depends on their method of production. If a liquid is used as the outermost focusing fluid, there may be dilution of the microcapsules due to the requirement for a relatively higher volumetric flow rate for the outermost fluid compared with the middle and inner fluids. As a result, in that case, it may be necessary to concentrate the microcapsules prior to their use in subsequent enzymatic reactions. Concentration may be achieved by, for example, filtration, centrifugation, or magnetic separation (e.g., if superparamagnetic particles are included inside the microcapsules by including them in the innermost fluid). If a gas is used as the outermost focusing fluid, the microcapsules may be collected by directing the jet from the aperture into an appropriate liquid bath (e.g., buffer), requiring less concentration prior to use. High speed flow sorting may be employed as an alternative or additional means of microcapsule concentration, additionally providing an efficient means of eliminating satellite particles, damaged microcapsules, or microcapsules exceeding the desired size.

The innermost flow may be any fluid comprising one or more enzymes (such as one or more polymerase enzymes) and nucleic acid template. In this embodiment of the invention, the enzyme can be any enzyme that alters a nucleic acid or that requires the use of a nucleic acid as a substrate. Examples of enzymes include, but are not limited to, polymerases (e.g., DNA polymerases, RNA polymerases), reverse transcriptases, ligases, Klenow fragment and restriction endonucleases.

In a preferred embodiment of the invention, the innermost fluid comprises a single, nucleic acid template. However, the innermost fluid can contain multiple copies of a single nucleic acid template. The innermost fluid may also comprise a buffer and assorted reagents for conducting the appropriate chemical reactions (e.g., nucleotides in the case of sequencing).

The middle fluid comprises an immiscible semipermeable membrane forming material. The middle fluid preferably comprises a monomer and/or reactive polymer that forms a semipermeable membrane, including, but not limited to, acrylic polymers (including, but not limited to, various acrylamides), cyanoacrylates, poly(ethylene glycol) diacrylates (PEG-DA), or poly(ethylene glycol) dimethacrylates (PEG-DMA) of various chain lengths and epoxy resins (including, but not limited to, DuPont's “Somos 6100” series of resins).

In certain cases, the semipermeable membrane forming material of the microcapsules is polymerized by chemical initiators included in the outermost fluid stream of the multiple sheath flow device. Examples of such chemical initiators include persulphate+3-dimethylaminopropionitrile (DMPAN), persulphate+tetramethylethylenediamine (TEMED), persulphate+heat, persulphate+thiosulfate, persulphate+bisulfite, persulphate+diethylmethylaminediamine (DEMED), H₂O₂+Fe², benzoyl peroxide, lauroyl peroxide, tetralin peroxide, actyl peroxide, caproyl peroxide, t-butyl hydroperoxide, t-butyl perbenzoate, t-butyl diperphthalate, cumene hydroperoxide, 2-butanone peroxide, azoinitiators, e.g. azodiisobutylnitrile and azodicarbonamide, riboflavin+visible light, methylene blue+a redox couple, and the like. Alternatively, the invention contemplates polymerizing the semipermeable membranes of the microcapsules by light exposure using appropriate photoinitiators.

The outer fluid comprises a liquid or gas capable of entraining the innermost liquid and middle semipermeable membrane forming material in the aperture of a multiple sheath flow device. Gases include inert gases such as nitrogen or argon so as to avoid inhibition of polymerization by oxygen. Liquids are preferably aqueous and may contain additives to control the rheological properties of the outermost sheath flow including viscosity, interfacial tension, and density.

Methods of the present invention include the use of a multiple sheath flow device for the preparation of a plurality of monodisperse microcapsules. By adjusting the relative flow rates of the three fluid feeds, it is possible to independently control the diameter of the microcapsules and the thickness of the encapsulating membrane. For instance, the diameter of the microcapsules can be adjusted by varying the Rayleigh instability of the jet and can range in size from 1 μm to several nm. In one embodiment of the invention, the microcapsules are approximately 1 to 10 μm in diameter. In another embodiment of the invention, a plurality of microcapsules comprise a diameter with a coefficient of variation of less than or about 10%.

The multiple sheath flow device can be operated at high pressure to increase the rate of microcapsule formation and minimize reagent consumption and can optionally be automated. For instance, the invention includes the use of computer controlled syringe pumps to inject the fluids (i.e., innermost liquid flow, semipermeable membrane forming material flow and outer fluid flow) into the multiple sheath flow device.

The multiple sheath flow device can be operated at a variety of microcapsule formation rates. In one method of the invention, the multiple sheath flow device forms microcapsules at a rate of at least about 50,000 microcapsules per second, at least about 100,000 microcapsules per second, at least about 500,000 microcapsules per second, or at least about 1,000,000 microcapsules per second.

### Microcapsules

Microcapsules produced by the methods of the present invention are useful for an enzyme-mediated reaction such as a polymerase-mediated reaction. For instance, the invention includes methods of producing a thermostable microcapsule that comprises a semipermeable membrane, an aqueous core, one or more polymerases in the aqueous core, and a nucleic acid template in the aqueous core. Microcapsules produced in accordance with the invention may be any appropriate shape or size, but a preferred microcapsule is spherical and approximately 1-10 μm in diameter. In one embodiment of the invention, the microcapsules are transparent and non-fluorescent.

The invention may be used to produce microcapsules that are thermostable and capable of withstanding thermocycling for PCR. By “thermostable”, it is meant that the microcapsule can withstand high temperatures such as those required to denature nucleic acids. Microcapsules of the invention can also be produced to withstand high-speed flow sorting, for instance, sorting at greater than about 70,000/second.

A multiple sheath flow can be used to produce a collection of microcapsules that are of relative uniform size, i.e., are monodisperse, and have a diameter with a coefficient of variation of less than or about 10%.

### Semipermeable Membrane

Ideally, the semipermeable membrane of a microcapsule according to the invention is impermeable to high molecular weight molecules. On the other hand, the semipermeable membrane is permeable to low molecular weight molecules such as low molecular weight reagents. For instance, the semipermeable membrane can be permeable to molecules possessing a molecular weight of less than or about 20,000 g/mol, less than or about 10,000 g/mol, less than or about 5,000 g/mol, or less than or about 3,000 g/mol. In one embodiment of the invention, the semipermeable membrane is impermeable to molecules with a molecular weight of about 3,000 g/mol or more and is permeable to molecules with a molecular weight molecule of less than about 3,000 g/mol.

Alternatively, the semipermeable membrane is impermeable to enzymes and nucleic acids that are longer than about 70 nucleotides in length. For instance, the semipermeable membrane is impermeable to the nucleic acid template contained within the aqueous core of the microcapsule.

The semipermeable membrane of each microcapsule is permeable to small molecular weight reagents and reaction byproducts. Thus, in sequencing, the semipermeable membrane is permeable to deoxynucleotide triphosphates (dNTPs), dideoxynucleotide triphosphates (ddNTPs), labeled ddNTPs, labels and dyes, pyrophosphates, divalent cations (e.g., magnesium ions and manganese ions), monovalent cations (e.g., potassium ions), and nucleic acids that are shorter than about 70 nucleotides in length.

The semipermeable membrane can comprise any polymer known in the art that is permeable to low molecular weight reagents and impermeable to high molecular weight reagents. It is important that the polymer not prevent an enzymatic reaction from occurring in the aqueous core of the microcapsule. Polymers that can be used, include, but are not limited to, acrylic polymers including, but not limited to various acrylamides, cyanoacrylates and poly(ethylene glycol) diacrylates (PEG-DA), poly(ethylene glycol) dimethacrylates and epoxy resins including DuPont's “Somos 6100” series of resins.

The semipermeable membrane can comprise a polymer that is capable of cross-linking In one embodiment of the invention, the polymer is a photocrosslinkable polymer. In one embodiment, crosslinking is used to control the molecular weight cut off (MWCO) of the membrane.

### Nucleic Acid Template

The nucleic acid template within the aqueous core of each microcapsule serves as the substrate for the enzyme-mediated reaction. In one embodiment of the invention, there is a single nucleic acid template, i.e., one molecule, in each microcapsule. For instance, in one embodiment of the invention, the microcapsule comprises a semipermeable membrane, an aqueous core, one or more polymerase enzymes in the aqueous core, and one nucleic acid template in the aqueous core. For instance, the present invention includes methods of producing a collection of microcapsules, wherein each microcapsule comprises a single nucleic acid template. The present invention also includes methods of producing a collection of microcapsules, wherein each microcapsule comprises a single, different nucleic acid template (i.e., the nucleic acid template is different for each microcapsule). In another embodiment of the invention, a multiple sheath flow device is used to produce microcapsules containing multiple copies of the same nucleic acid templates.

The nucleic acid template can be either a DNA template or an RNA template, including genomic DNA, cDNA, PNA, mRNA, rRNA, tRNA, gRNA, siRNA, micro RNA, and others known in the art.

As can be appreciated by a skilled artisan, nucleic acid template can be a eukaryotic, prokaryotic, or viral nucleic acid; and may be a recombinant nucleic acid or a previously amplified nucleic acid fragment (e.g., a PCR product).

Although the nucleic acid template may be derived from a clone, it is unnecessary to clone the nucleic acid molecule in vivo prior to use in the microcapsule. For instance, sheared or enzymatically digested genomic nucleic acids may be used as nucleic acid templates.

The nucleic acid template can vary in length so long as it is of sufficient size to prevent it from crossing the semipermeable membrane. For instance, the nucleic acid template can be about or greater than 100 nucleotides in length, about or greater than 200 nucleotides in length, about or greater than 300 nucleotides in length, about or greater than 400 nucleotides in length, about or greater than 500 nucleotides in length, about or greater than 600 nucleotides in length, about or greater than 700 nucleotides in length, about or greater than 800 nucleotides in length, about or greater than 900 nucleotides in length, about or greater than 1000 nucleotides in length, about or greater than 1100 nucleotides in length, or about or greater than 1200 nucleotides in length. The invention includes microcapsules comprising a nucleic acid template that is at least about 100 nucleotides in length, at least about 200 nucleotides in length, at least about 300 nucleotides in length, at least about 400 nucleotides in length, at least about 500 nucleotides in length, at least about 600 nucleotides in length, at least about 700 nucleotides in length, at least about 800 nucleotides in length, at least about 900 nucleotides in length, at least about 1000 nucleotides in length, at least about 2000 nucleotides in length, at least about 3000 nucleotides in length, at least about 4000 nucleotides in length, or at least about 5000 nucleotides in length.

The nucleic acid template may be either linear or circular, the circular topology having the added benefit of a reduced tendency to penetrate the polymer shell of the microcapsule.

The nucleic acid template must also contain at least one priming site for hybridization of a complementary primer oligonucleotide for DNA amplification. The nucleic acid template may be single-stranded or double-stranded, although the preferred template is single-stranded.

### Enzymes

Microcapsules prepared by the methods of the present invention comprise one or more enzymes in an aqueous core. Examples of enzymes include nucleic acid modifying enzymes such as polymerases, reverse transcriptases, ligases, Klenow fragment and restriction endonucleases. Examples also include thermophilic DNA polymerases such as Taq DNA polymerase I, DNA polymerase II, DNA polymerase III holenzyme, DNA polymerase IV, terminal transferase, Klenow fragment, T4 DNA polymerase, T7 DNA polymerase, and phi29 DNA polymerase. Additional examples of enzymes include various forms of “hot start” polymerases that are inactive at low temperature (e.g., 40° C.) and only become active upon heating to relatively high temperatures (e.g., >90° C.).

In another embodiment of the invention, the enzymes are selected from RNA polymerase I, RNA polymerase II, RNA polymerase III, and T7 RNA polymerase.

Microcapsules produced by the methods of the invention may contain more than one enzyme within an aqueous core. For instance, the present invention includes microcapsules with one, two, three, or four or more different enzymes within the aqueous core.

### Low Molecular Weight Reagents and Buffers

Microcapsules produced by the methods of the present invention are permeable to low molecular weight reagents and buffers. Microcapsules further comprise one or more low molecular weight reagents. In one embodiment of the invention, the aqueous core is a buffer solution. Microcapsules are stored or incubated in a solution comprising low molecular weight reagents and/or a buffer solution. Examples of low molecular weight reagents are described throughout this application and include dNTPs, ddNTPs, labeled ddNTPs (e.g., fluorescently labeled ddNTPs), divalent cations, monovalent cations, stabilizers and nucleic acid primers. Buffers that can be used with the microcapsules of the invention include polymerase buffers such as standard Taq buffer.

### Primers

Microcapsules prepared by methods of the present invention may be used with primers of any size. In other words, the microcapsules can be used with primers that are capable of passing through the membrane of the microcapsule as well as those are incapable of passing through the membrane of the microcapsule.

In one embodiment of the invention, the primers are able to pass through the semipermeable membrane of the microcapsule. Such primers can be up to about 70 nucleotides in length. For instance, primers that are about 5 to 10 nucleotides in length, 10 to 15 nucleotides in length, 10 to 20 nucleotides in length, 10 to 30 nucleotides in length, 15 to 25 nucleotides in length, or 25 to 30 nucleotides in length can be used with the microcapsule of the invention as well as primers that are about 40 or fewer nucleotides in length, about 50 or fewer nucleotides in length, about 60 or fewer nucleotides in length, and about 70 or fewer nucleotides in length. In one embodiment of the invention, the primers are about 20 to 50 nucleotides in length.

In another embodiment of the invention, each microcapsule contains one or more primers that are unable to diffuse out of the aqueous core due to size. As can be appreciated by a skilled artisan, the size of the primers can vary depending on the polymer used as a semipermeable membrane. However, generally, primers greater than about 70 nucleotides are unable to cross the semipermeable membrane of the microcapsule.

The primers are substantially complementary or perfectly complementary to a region of the nucleic acid template. In one embodiment, the primer contains a small number of mismatches compared to the nucleic acid template that do not interfere with the ability of the primer to anneal to the nucleic acid template under stringent conditions. Such a primer may contain 5 or fewer, 4 or fewer, 3 or fewer, 2 or fewer, or 1 mismatches compared to the nucleic acid template.

In one embodiment, universal primers can be used to hybridize with a common motif in the template. For example, the primer can be a poly-T primer that is capable of binding to a poly-A region in a template nucleic acid. Random primers are, of course, also useful.

The amplification reaction can be a PCR reaction (including rtPCR and others as described below) or can be another type of amplification, such as rolling circle amplification. Rolling circle amplification is described in U.S. Pat. Nos. 6,576,448; 6,977,153; and 6,287,824, each of which is incorporated by reference herein.

### Methods of Using Microcapsules for Amplification of Nucleic Acids

Nucleic acid templates can be amplified in microcapsules of the invention. As can be appreciated by a skilled artisan, various nucleic acid amplification methods known in the art that employ an enzyme-mediated reaction can be easily modified for use with the present invention. The only requirement is that the one or more enzymes, nucleic acid template to be amplified, and, optionally, primers, be encapsulated within the semipermeable membrane of the microcapsule. For instance, in addition to polymerase chain reaction (PCR), other known amplifications methods such as rolling circle amplification (RCA), ligase chain reaction (European application EP 320 308), gap filling ligase chain reaction (U.S. Pat. No. 5,427,930), strand displacement amplification (U.S. Pat. No. 5,744,311) and repair chain reaction amplification (WO 90/01069) may be performed using the microcapsules of the present invention.

In one embodiment of the invention, microcapsules are used to amplify a nucleic acid template by polymerase chain reaction. In this case, the microcapsule contains a thermostable polymerase (e.g., Taq polymerase) and a nucleic acid template. Preferably, the aqueous core and liquid surrounding the microcapsule contain a PCR buffer solution and dNTPs. Primers that are complementary to the nucleic acid template may be located within the aqueous core or, if the capable of traversing the semipermeable membrane, in the PCR buffer solution bathing the microcapsule.

To perform PCR, one to over a billion microcapsules are placed in a tube with the appropriate PCR reagents. As with traditional PCR, the tube is placed in a thermocycler under conditions necessary for PCR (i.e., cycles of denaturation, annealing, and elongation). Briefly, in a thermocycler, the microcapsules are denatured by heating (e.g., 94° C. to 98° C.) for about 20 to 30 seconds. The microcapsules are then subjected to an annealing temperature (e.g., about 50° C. to 65° C.) for about 20 to 40 seconds. Elongation proceeds next. The elongation temperature (usually about 72° C. to 80° C.) and time (about 1000 bases/minute) required for the elongation step depend on the polymerase enzyme used and length of nucleic acid template, respectively. The denaturation, annealing, and elongation steps are repeated several times (usually about 20 to 30 cycles) and may be capped off with an extended elongation step.

One or multiple nucleic acid templates may be amplified in a single reaction using one or a plurality of microcapsules. In one embodiment of the invention, each microcapsule contains multiple nucleic acid templates that are amplified by PCR (i.e., multiplex PCR). In a preferred embodiment of the invention, each microcapsule contains a single nucleic acid template that is amplified by PCR. In another preferred embodiment, billions of microcapsules, each microcapsule containing a single nucleic acid template, are amplified by PCR.

In a preferred embodiment, microcapsules are used to amplify a nucleic acid template by rolling circle amplification (RCA). In that case, the microcapsule contains a strand displacement DNA polymerase (e.g., phi29 polymerase) and a circular nucleic acid template for amplification. Preferably the aqueous core and liquid surrounding the microcapsule contain a RCA buffer solution and dNTPs. A primer that is complementary to the circular nucleic acid template may be located within the aqueous core or, if capable of traversing the semipermeable membrane, in the RCA buffer solution bathing the microcapsule.

To perform RCA, one to over a billion microcapsules are placed in a tube with the appropriate RCA reagents. Isothermal amplification (e.g., 40° C.) of the circular template results in a linear concatamer of very high molecular weight that does not cross the polymer membrane of the capsule.

Microcapsules produced by methods of the present invention can also be used in reverse transcriptase amplification reactions. In this embodiment of the invention, each microcapsule comprises a semipermeable membrane, an aqueous core, a reverse transcriptase and an RNA template for amplification.

### Methods of Using Microcapsules for Sequencing of Nucleic Acids

In one embodiment of the invention, a starting nucleic acid template is amplified within a microcapsule as described above prior to sequencing. Although not necessary, one or more microcapsules that have previously been subjected to an amplification reaction can be “cleaned-up” prior to sequencing by dialyzing the microcapsules in a suitable buffer.

In one embodiment, microcapsules that have previously undergone amplification are used in a Sanger sequencing reaction. Depending on the number of templates to be sequenced, one to thousands, even millions or billions, of microcapsules are placed in a tube with sequencing reagents. Sequencing reagents include dNTPs, ddNTPs and primers. Preferably, the ddNTPs are fluorescently labeled so that all four ddNTPs can be incorporated in the growing DNA chain in a single reaction.

Depending on the desired read length of the sequencing reaction (e.g., 1,000 bases) and the sensitivity requirements of the fluorescence detection system (e.g., 1,000 labeled fragments per band), then the total number of Sanger extension products can be estimated (e.g., 1,000×1,000=1 million). If the initial single molecule template in each capsule has already been amplified to an equivalent number of copies (e.g., 1 million), then only a single cycle of polymerase extension and ddNTP termination will be required to produce the required number of Sanger extension products. If, however, the initial single molecule template in each microcapsule has been amplified to a lesser extent, then multiple cycles of polymerase extension and ddNTP termination can be employed using cycle sequencing to generate the necessary number of extension products. Cycle sequencing is performed in a thermocycler by methods known in the art.

Upon completion of the sequencing reaction, it is preferable that unincorporated ddNTPs, dNTPs, primers and pyrophosphates are removed. In one embodiment of the invention, unwanted reagents and byproducts are removed by dialysis against a suitable buffer.

### Flow Sorting

In one embodiment of the invention, it is preferred that each microcapsule contains a single starting nucleic acid template. Poisson statistics dictate the dilution requirements needed to insure that each microcapsule contains only a single starting nucleic acid template. For example, if, on average, each microcapsule is to contain only a single template, about ⅓ of the microcapsules will be empty and contain no nucleic acid template, about ⅓ will contain exactly one nucleic acid template, and about ⅓ will contain two or more templates.

The microcapsule population may be enriched to maximize the fraction that started with a single nucleic acid template. Because the Sanger sequencing reaction incorporates fluorescently labeled ddNTPs, it is possible to flow sort the microcapsules after sequencing (and, preferably, after a purification step) to enrich for those that are fluorescent rather than empty. High speed flow sorters, such as the MoFlo™ (Beckman-Coulter, Inc.), are capable of sorting at rates in excess of 70,000 per second and can be used to enrich a population of microcapsules of the invention. Similarly, it is possible to exploit other differences between full and empty microcapsules (e.g., buoyant density) to enrich a population of microcapsules. In order to enrich for microcapsules with one starting nucleic acid template as opposed to several different starting templates, it may be desirable to skew the Poisson distribution accordingly.

### Sequence Detection Methods

Electrophoresis-based sequencers and non-electrophoresis-based sequencers can be used to determine the sequence of the nucleic acid product contained within each of the microcapsules. In one embodiment of the invention, microcapsules are loaded either manually or automatically into the sequencer and broken in situ to release the contents of the microcapsules. In another embodiment of the invention, microcapsules are loaded manually or automatically into the sequencer without the need for breaking the microcapsules.

Electrophoresis methods, including slab gel-based, capillary-based, and microchip based methods known in the art, can be used to determine the sequence of the one or more microcapsules. Capillary-based methods include commonly used sequencers such as the Applied Biosystems 3730 Series DNA analyzers as well as newer massively parallel continuous electrophoresis systems.

The microcapsules described herein can be used to perform thousands, even millions or billions, of Sanger sequencing reactions in a single tube. In one preferred embodiment of the invention, a massively parallel electrophoresis system is used to read each sequence. In one embodiment of the invention, microcapsules are loaded or the sequenced product from microcapsules are loaded on to a continuous film electrophoresis sequencer for sequence determination.

In yet another embodiment of the invention, the sequence of the nucleic acid of one or more microcapsules is determined in real time (i.e., sequencing-by-synthesis). In this embodiment, the sequencing reaction and determination of the sequence occur simultaneously.

### Kits Containing Microcapsules

In a further embodiment, the present invention provides kits containing microcapsules. In one embodiment, the kit comprises a container with a label. Suitable containers include, for example, bottles, vials, and test tubes. The containers may be formed from a variety of materials such as glass or plastic. The container holds a composition that includes a microcapsule or plurality of microcapsules as described herein.

A kit of the invention will typically comprise the container described above and one or more other containers comprising materials desirable from a commercial end user standpoint, including, but not limited to, buffers, reagents, and package inserts with instructions for use. In one embodiment of the invention, the kit contains dNTPs and/or ddNTPs.

### EXAMPLE 1

The following example demonstrates an embodiment of the manufacture and use of microcapsules according to the invention.

Three model NE500 syringe pumps (New Era Pump Systems, Inc., Wantagh, N.Y.) controlled by a PC running WinPumpControl software (Open Cage Software, Inc., Huntington, N.Y.) deliver fluids to the flow focusing nozzle inlet fittings illustrated in FIG. 1. An appropriately sized Luer-Lok® syringe is mounted on each pump and connected to the flow focusing nozzle by PEEK capillary tubing (Upchurch Scientific, Oak Harbor, Wash.). The pinhole aperture in the flow focusing nozzle is a model RB 22824 sapphire orifice (Bird Precision, Inc., Waltham, Mass.). The cylindrical portion of the orifice is 235 μm in diameter and 533 μm long. The innermost flow focusing tube delivering the Core Solution to be encapsulated is made of PEEK with an ID of 150 μm and an OD of 360 μm. This innermost tube is centered in a second PEEK capillary tube with an ID of 762 μm and an OD of 1587 μm, delivering the Polymer Shell Solution as a surrounding coaxial flow through the annular gap between the tubes. The exit end of the innermost tube is recessed by 500 μm from the exit tip of the surrounding tube, which is positioned at a height of 500 μm and centered on the orifice. The Focusing Solution is provided as a third coaxial flow through the annular gap between the machined body of the flow focusing nozzle and the outer capillary tube.

To form impermeable polymer shell microcapsules, the following three solutions were delivered to the flow focusing nozzle at the indicated volumetric flow rates: (1) Core Solution—sodium fluorescein (5 mg/mL—Fluka/Sigma-Aldrich, St. Louis, Mo.), glycerin (25% v/v—Walgreens, Deerfield, Ill.) in distilled water at 0.1 mL min⁻¹; (2) Polymer Shell Solution—PEGDMA 200 (polyethylene glycol 200 dimethacrylate) (Monomer-Polymer & Dajak Labs, Inc., Feasterville, Pa.), 4.76% v/v 2-hydroxy-2-methyl propiophenone (Sigma-Aldrich, St. Louis, Mo.), 0.4% v/v TEMED (N,N,N′,N′-tetramethylethylenediamine) and 0.05 g/mL 2,2-dimethoxy-2-phenyl-acetophenone (Sigma-Aldrich, St. Louis, Mo.) at 0.15 ml min⁻¹; and (3) Focusing Fluid—1% poly(vinyl alcohol) 87-89% hydrolyzed (Typical M_{w} 85,000-124,000) (Sigma-Aldrich, St. Louis, Mo.) in distilled water at 6.5 mL min⁻¹. The orifice of the flow focusing nozzle is positioned ˜15 cm above the liquid surface of a 100 mL beaker containing 50 mL of the Focusing Fluid to collect the microcapsules. The beaker sits on a near-UV transilluminator (Spectroline Slimline™ Series 365 -8W, Spectronics Corporation, Westbury, N.Y.) to provide 360 nm illumination for photoinitiation. In addition, two 40W “black light” fluorescent lamps (GE F40BLB—General Electric Company, Fairfield, Conn.) are positioned ˜10 cm to the side of the emerging liquid jet to photoinitiate polymerization of the shell of the microcapsule prior to “splash down” in the collection beaker. Aliquots (35 μL) of microcapsules are mounted on a microscope slide and examined in an Axiovert 25 inverted microscope (Carl Zeiss Microlmaging, Inc., Thornwood, N.Y.). Digital images are captured using an AxioCam CCD camera (Carl Zeiss Microlmaging, Inc., Thornwood, N.Y.) and analyzed using AxioVision software Ver 4.6 (Carl Zeiss Microlmaging, Inc., Thornwood, N.Y.). Fluorescence imaging employs a 50 W Hg lamp illuminator (Carl Zeiss Microlmaging, Inc., Thornwood, N.Y.) and a FITC filter cube (484 nm excitation, 494/521 nm emission) (Carl Zeiss Microlmaging, Inc., Thornwood, N.Y.). Example bright field and fluorescence images of impermeable polymer shell microcapsules generated using the above described system are provided in FIGS. 5A and 5B. Encapsulation efficiency of the fluorescein labeled Core Solution is estimated at 99% with a mean microcapsule diameter of 90 μm±15 μm and a shell thickness of 5 μm. Microcapsule formation rate is estimated at ∫11,000 sec⁻¹. Microcapsules examined immediately after polymerization are indistinguishable from those placed in distilled water for up to several weeks at room temperature, indicating no loss of fluorescein. The polymer shells of these microcapsules are therefore impermeable to fluorescein (M_{R} 376).

### EXAMPLE 2

Smaller diameter impermeable polymer shell microcapsules are generated by adjusting the relative flow rates of the solutions from Example 1 as follows: Core Solution—0.025 ml min⁻¹; Polymer Shell Solution—0.05 ml min⁻¹; and Focusing Fluid—6.0 ml min⁻¹. The resulting microcapsules, illustrated in FIG. 6, are <10 μm in diameter.

### EXAMPLE 3

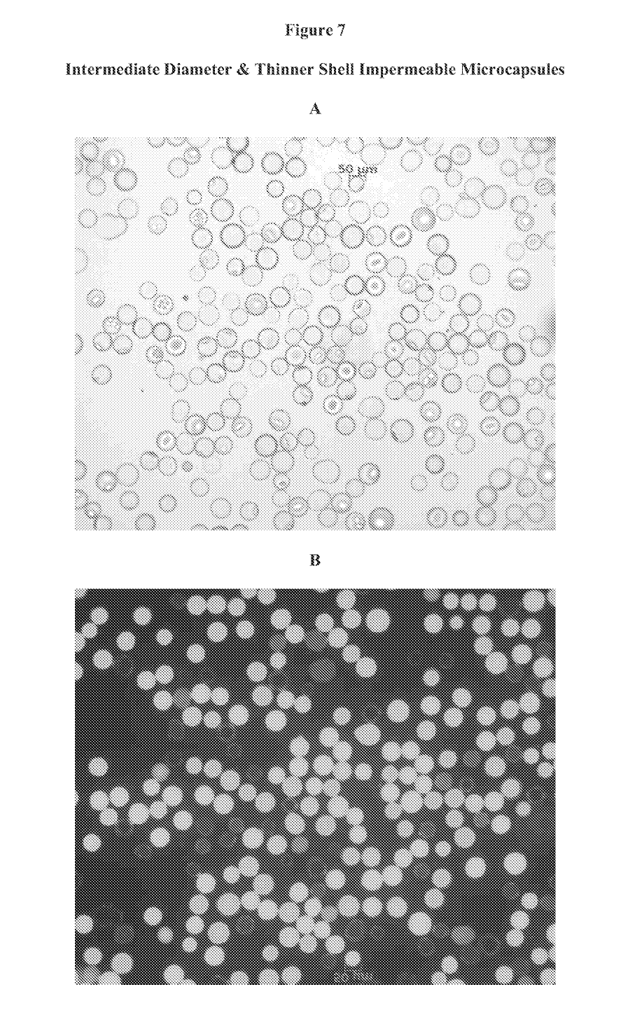

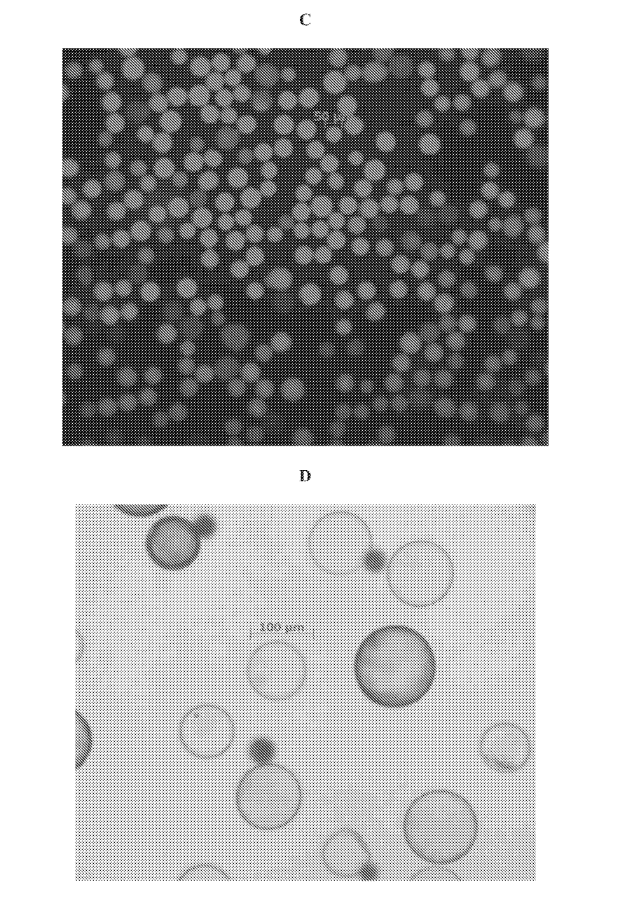

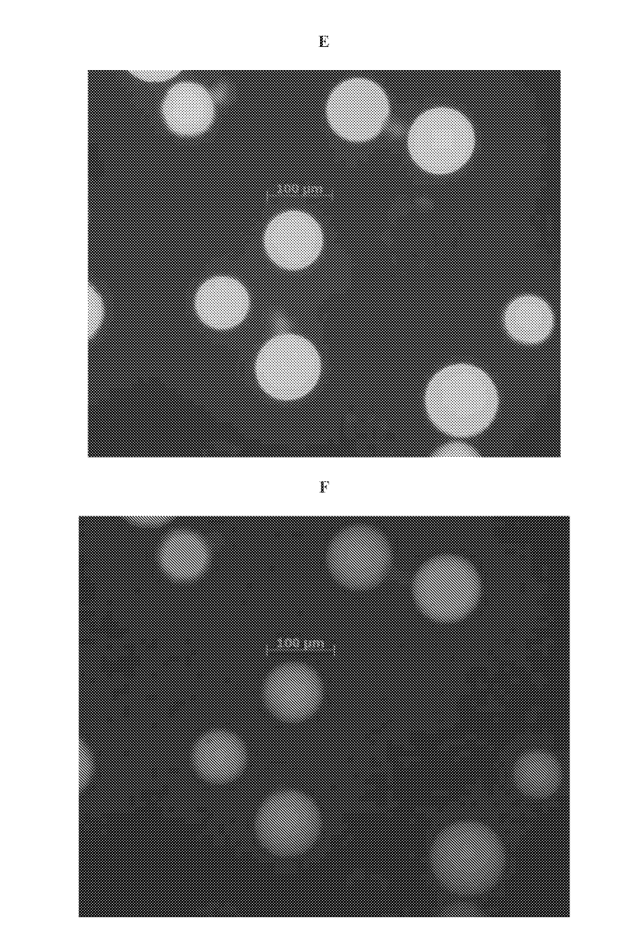

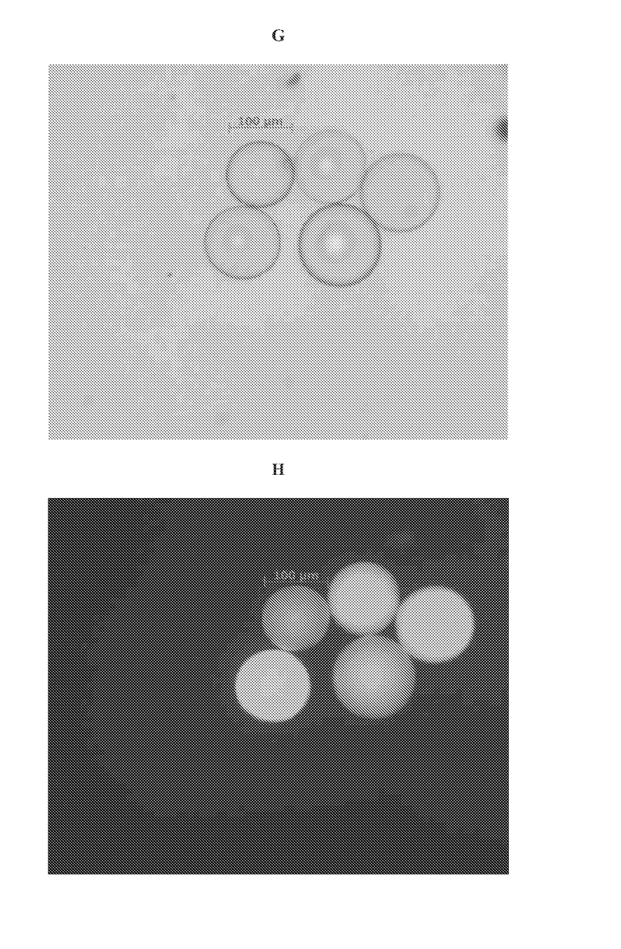

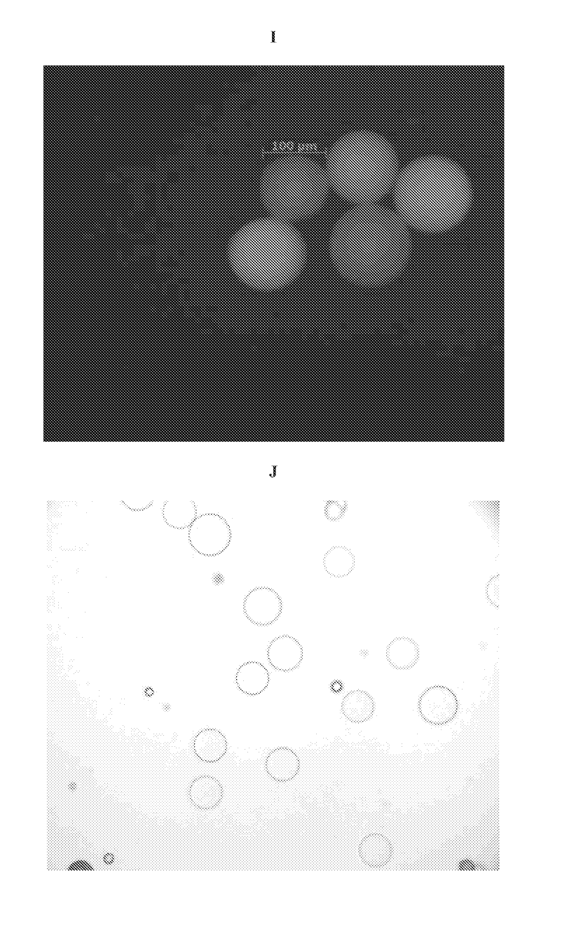

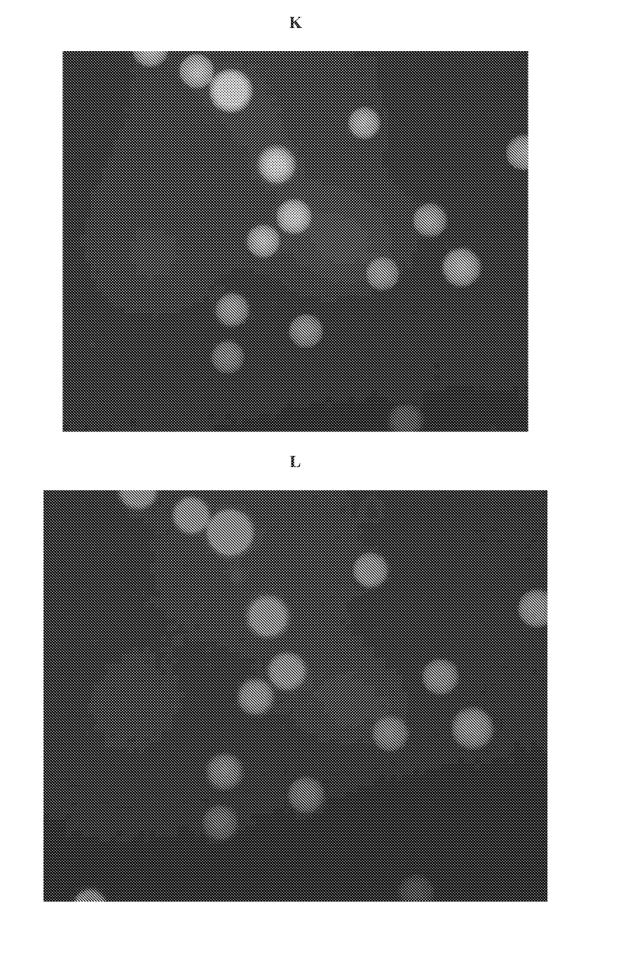

Intermediate diameter and/or thinner shell impermeable polymer microcapsules can also be produced using an alternative Polymer Shell Solution, blending PEGDMA 200 with PEGDA (poly(ethylene glycol) diacrylate of different chain lengths (PEGDA575-Mₙ ˜575 or PEGDA700—Mₙ˜700—Sigma-Aldrich, St. Louis, Mo.) in the ratio of 4:1 PEGDMA 200:PEGDAXXX and by adjusting the relative flow rates of the three solutions The Core Solution is composed of a low molecular weight fluorescent marker (sodium fluorescein M_{w}=376 kDa) and a high molecular weight marker (rhodamine B isothiocyanate-labeled dextran M_{w}=10 kDa) loaded together into the microcapsules in approximately equimolar amounts using the following composition: sodium fluorescein (0.26 mg/mL—Fluka/Sigma-Aldrich, St. Louis, Mo.) and rhodamine B isothiocyanate-dextran (5 mg/mL—Sigma-Aldrich, St. Louis, Mo.) and glycerol (25% v/v—Sigma, St. Louis, Mo.) in distilled water. Intermediate size microcapsules measuring ˜50 μm in diameter were produced using PEDGA575 with the following flow rates: Core Solution ˜0.05 ml min⁻¹, Polymer Shell Solution—0.010 ml min⁻¹, and Focusing Fluid—15 ml min⁻¹. The resulting microcapsules are shown in FIG. 7A-C. Progressively thinner polymer shells were produced using PEGDA700 by reducing the Polymer Shell Solution flow rate from 0.10 (FIGS. 7D-F) to 0.08 (FIGS. 7G-I) to 0.05 ml min⁻¹ (FIGS. 7J-L) while keeping the Core Solution and Focusing Fluid flow rates contant at 0.10 ml min⁻¹ and 6.5 ml min⁻¹ respectively.

### EXAMPLE 4

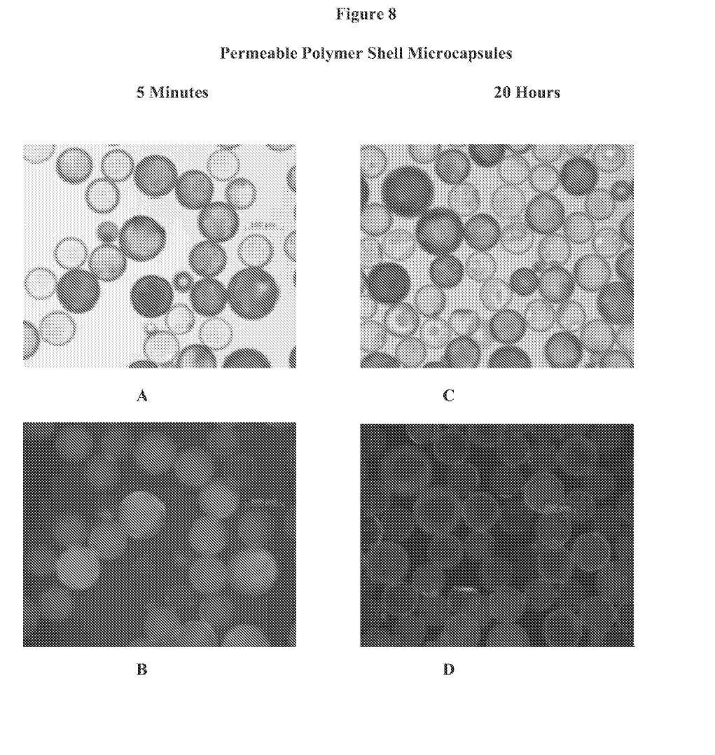

Permeable microcapsules are generated under identical conditions to Example 1 except for the addition of 5% v/v acrylic acid (Sigma-Aldrich, St. Louis, Mo.) to the Polymer Shell Solution as shown in FIGS. 8A-D. Encapsulation efficiency, microcapsule diameter and shell thickness are identical to the impermeable microcapsules, but display a darker and rougher appearance. Microcapsules imaged 5 minutes after formation display fluorescein content similar to that of the impermeable capsules, but when imaged after 20 hour incubation in distilled water at room temperature, the microcapsules have lost most of their fluorescein content while retaining their intact shell morphology, providing evidence of their permeability to fluorescein (M_{R} 376).

### EXAMPLE 5

Semi-permeability of the polymer shell of the microcapsules as produced in Example 4 was demonstrated by comparing the relative loss/retention of a low molecular weight fluorescent marker (sodium fluorescein M_{w}=376 Da) and a high molecular weight marker (rhodamine B isothiocyanate-labeled dextran M_{w}=10 kDa) loaded together into the semi-permeable microcapsules in approximately equimolar amounts as described in Example 3. All conditions were identical to those in Example 4, except for the composition of the Core Solution, which was modified as follows: sodium fluorescein (0.26 mg/mL—Fluka/Sigma-Aldrich, St. Louis, Mo.) and rhodamine B isothiocyanate-dextran (5 mg/mL—Sigma-Aldrich, St. Louis, Mo.) and glycerol (25% v/v—Sigma, St. Louis, Mo.) in distilled water. Impermeable micorcapsules were prepared as controls using conditions identical to those provided in Example 1 with the Core Solution detailed above.

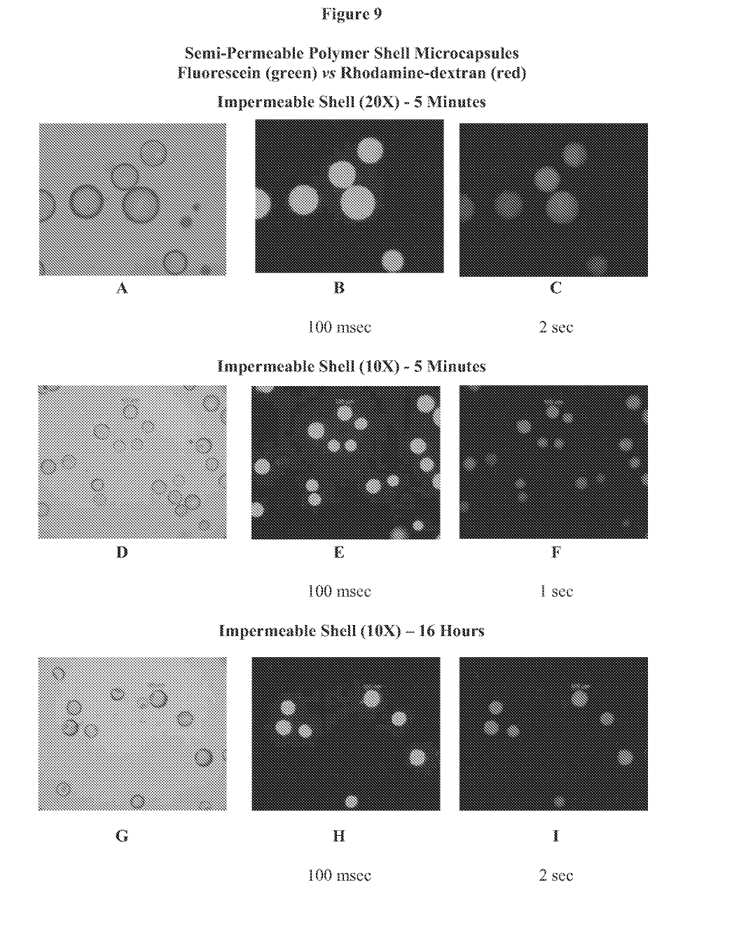

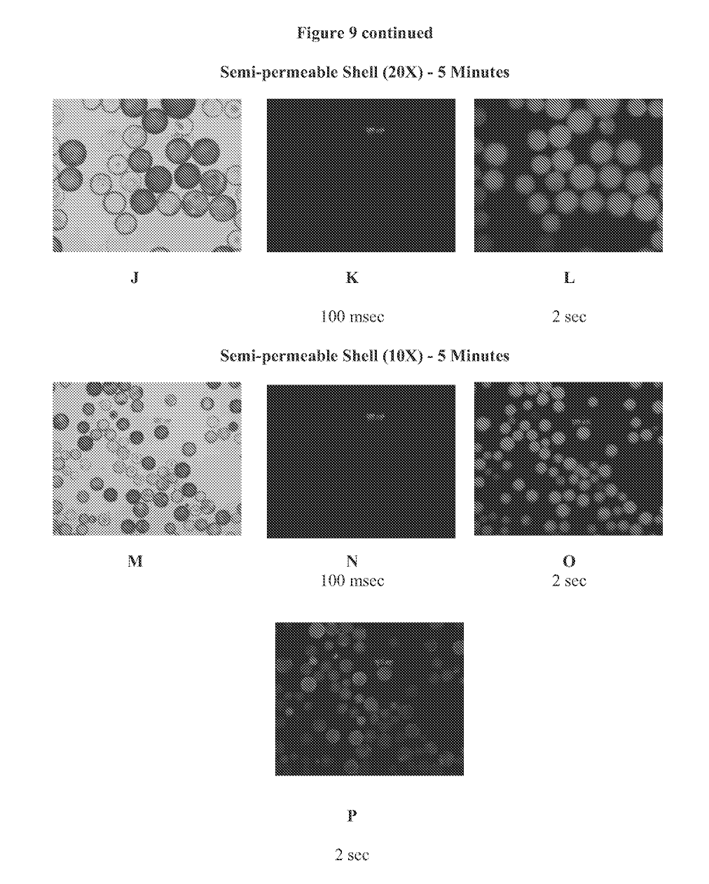

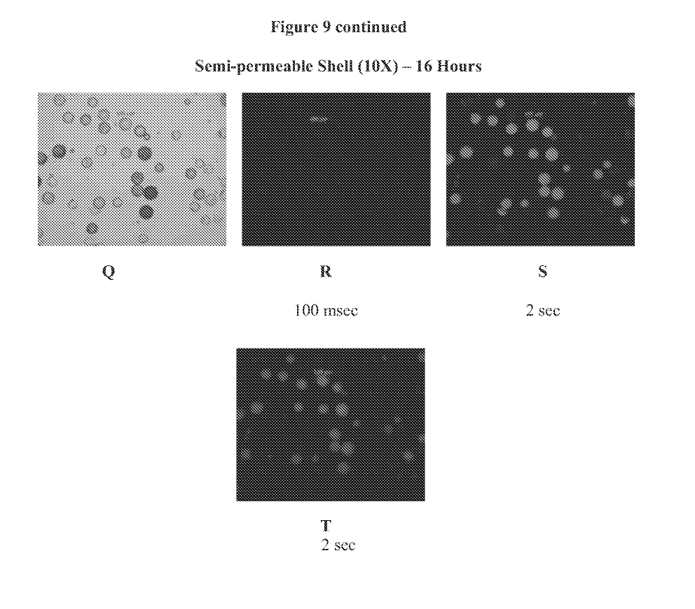

The harvested microcapsules were imaged directly in Focusing Fluid without washing ˜5 minutes after they were created. The microcapsules were then stored at room temperature in Focusing Fluid for ˜16 hours and reimaged. Brightfield and fluorescence images are provided in FIG. 9 below. Exposure times are indicated below each fluorescent image.

There was substantial loss of fluorescein within 5 minutes from the semi-permeable capsules compared with the impermeable control microcapsules, while there was no obvious loss of the rhodamine-labeled dextran even after 16 hours in the semi-permeable microcapsules, indicating that these microcapsules were preferentially permeable to the lower molecular weight fluorescein while retaining the higher molecular weight rhodamine-labeled dextran polymer. The Molecular Weight Cut Off (MWCO) of the semipermeable polymer shell membrane of these microcapsules is therefore >400 Daltons but <10,000 Daltons.

### EXAMPLE 6

Altered permeability characteristics of polymer shell microcapsules were demonstrated as described in Example 2 using an alternative Polymer Shell formulation. All conditions were identical, except for the composition of the Core Solution, which was modified as follows: fluorescein isothiocyanate-dextran (2 mg/mL—Fluka/Sigma-Aldrich, St. Louis, Mo.) and rhodamine B isothiocyanate-dextran (5 mg/mL—Sigma-Aldrich, St. Louis, Mo.) and glycerol (25% v/v—Sigma, St. Louis, Mo.) in distilled water, and the Polymer Shell Solution, which was modified as follows: 2:1 v/v PEGDMA 200 and MPEOEA (methoxypoly(ethyleneoxy) ethyl acrylate) (Monomer-Polymer & Dajak Labs, Inc., Feasterville, Pa.).

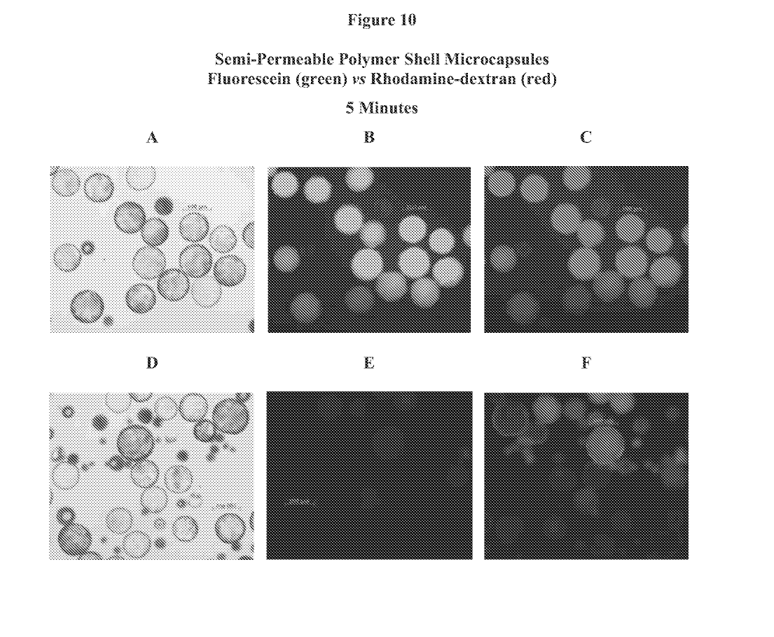

The harvested microcapsules were imaged directly in Focusing Fluid without washing ˜5 minutes after they were created. The microcapsules were then stored in the dark at room temperature in Focusing Fluid for ˜24 hours and reimaged. Brightfield and fluorescence images are provided in FIG. 10. Exposure times are indicated below each fluorescent image.

There was no significant loss of either fluorescent signal from the permeable capsules within 5 minutes compared with the impermeable control microcapsules. However, at t=24 hrs, both signals had decreased significantly with this polymer shell formulation. The Molecular Weight Cut Off (MWCO) of the permeable polymer shell membrane of these microcapsules is therefore >10,000 Daltons.

### EXAMPLE 7

Semi-permeability of the polymer shells of the microcapsules was further demonstrated by encapsulation of high molecular weight DNA (single-stranded M13mp18 DNA—M_{w} 2.4 MDa, 7,249 bases) in the microcapsules and then labeling the DNA inside the microcapsules by incubating them in an exogenously added, low molecular weight fluorescent dye specific for single-stranded DNA (OliGreen®, M_{w}<1,000 Da—Invitrogen/Molecular Probes, Eugene, Oreg.). Semi-permeable microcapsules were produced as described in Example 5. All conditions were identical, except for the composition of the Core Solution, which was modified as follows: single-stranded M13mp18 DNA (20 μg/mL—Sigma-Aldrich, St. Louis, Mo.) in 1× TE buffer (10 mM Tris (TRIZMA®—tris(hydroxymethyl)aminomethane hydrochloride—Sigma-Aldrich, St. Louis, Mo.), 1 mM EDTA (ethylenediamenetetraacidic acid—Sigma-Aldrich, St. Louis, Mo.), pH 8.1) containing glycerol (25% v/v—Sigma-Aldrich, St. Louis, Mo.), and the Polymer Shell Solution, which was modified as follows: 10:1 v/v PEGDMA 200 and MPEOEA (methoxypoly(ethyleneoxy) ethyl acrylate) (Monomer-Polymer & Dajak Labs, Inc., Feasterville, Pa.). Negative control microcapsules were made with a Core Solutions containing only TE buffer and glycerin.

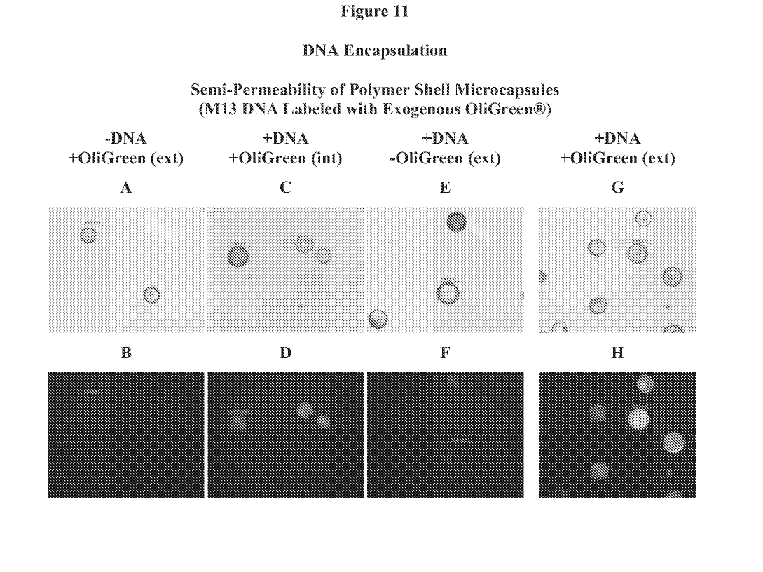

The harvested microcapsules were decanted and rinsed 2× with 20 mL distilled water. Negative control microcapsules without DNA and microcapsules containing the DNA Core Solution were incubated by mixing 100 μL of microcapsule suspension with 40 μL of a 1:20 dilution of OliGreen® in TE buffer. Fluorescence images were taken after 1 hour of incubation in the dark at room temperature, and are provided in FIG. 11. Exposure time for all images: t=5 secs.

There was no observable fluorescence from OliGreen® when added exogenously to negative control microcapsules without DNA. Microcapsules containing 20 μg/mL of single-stranded M13 DNA were brightly stained after incubation for 1 hour in exogenously added OliGreen®, indicating that these microcapsules were preferentially permeable to the lower molecular weight OliGreen® dye while retaining the much higher molecular weight single-stranded M13 DNA. The Molecular Weight Cut Off (MWCO) of the semi-permeable polymer shell membrane of these microcapsules is therefore >1,000 Daltons but <2.4 million Daltons.

### EXAMPLE 8

DNA amplification in semi-permeable polymer shell microcapsules was demonstrated using hyperbranched Rolling Circle Amplification (RCA). High molecular weight DNA (single-stranded M13mp18 DNA—M_{w} 2.4 MDa, 7,249 bases) was incorporated in microcapsules along with φ29 polymerase, random hexamers as primers, and deoxynucleotide triphosphate mix (dNTP mix). Semi-permeable microcapsules were produced as described in Example 4. All conditions were identical, except for the composition of the Core Solution, which was modified as follows: RCA Mix formulated by combining 13.5 μL diluted single-stranded M13mp18 DNA (1 μg/mL—Sigma-Aldrich, St. Louis, Mo.), 2.125 μL concentrated φ29 polymerase in buffer (New England Biolabs, Ipswich, Mass.), 8.75 μL random hexamer primers in H₂O (New England Biolabs, Ipswich, Mass.), 8.75 μL glycerol (25% v/v—Sigma-Aldrich, St. Louis, Mo.), 3.75 μL 10× RCA buffer (37 mM TRIS-HCl, 50 mM KCl, 10 mM MgCl₂, 5 mM NH₂SO₄, 1 mM DTT (dithiothrietol), 1×BSA), 0.4 μL 10× BSA (bovine serum albumin—New England Biolabs, Ipswich, Mass.) and 3.75 μL dNTP mix (New England Biolabs, Ipswich, Massachusetts), and the Focusing Fluid, which was composed of 5 wt % PVA in 1× RCA buffer. DNA cannot be visually detected at this low initial concentration in polymer microcapsules.

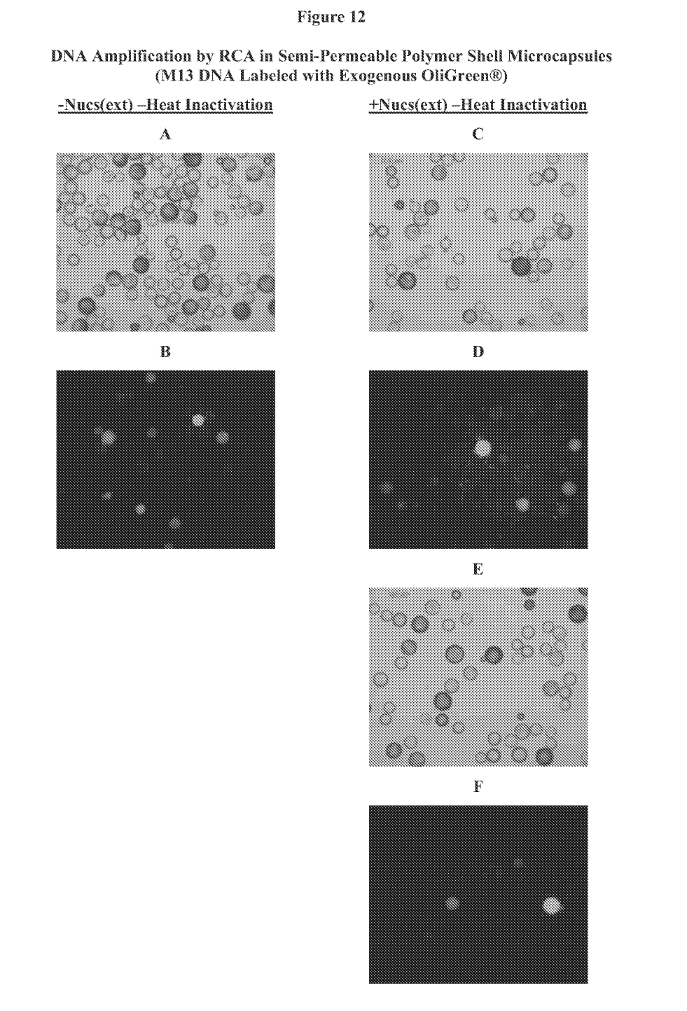

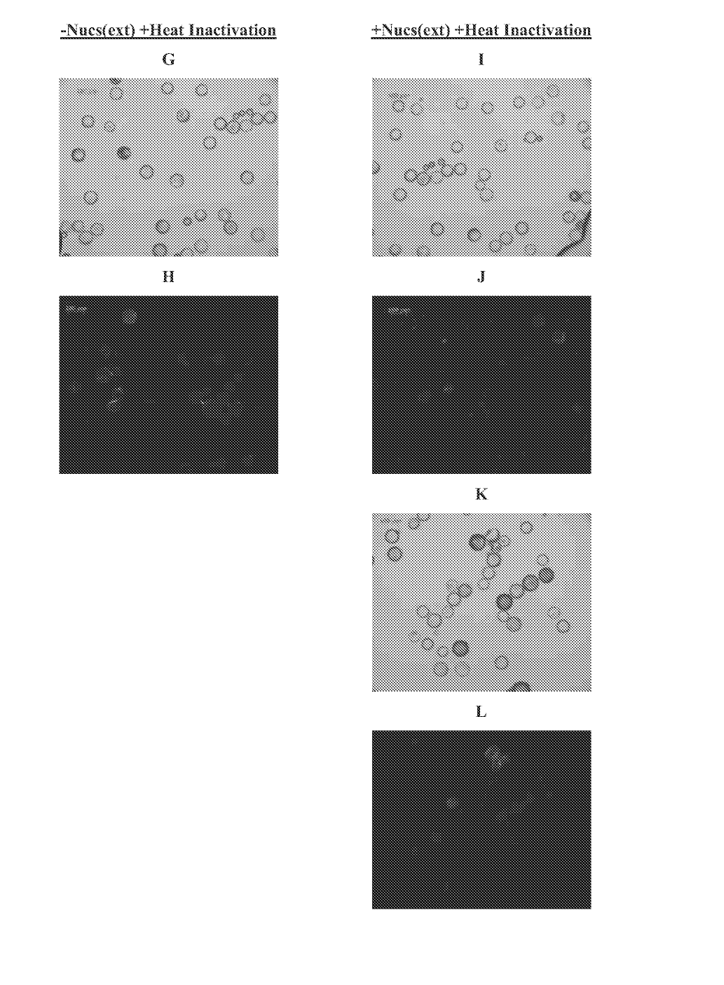

The harvested microcapsules were split into four 100 μL batches in 500 μL Safe-Lock Eppendorf microfuge tubes (Brinkmann Instruments, Inc., Westbury, N.Y.). The first batch was incubated as described below with no further treatment. This polymer microcapsule formulation is known to be permeable to dye molecules that are approximately the same molecular weight as native nucleotides. Therefore, 25 μl dNTP mix was added externally to the second and fourth batches. The third and fourth batches were then subjected to a five minute heat inactivation of the φ29 polymerase at 65° C. The four microfuge tubes containing the microcapsules were incubated at 30° C. for 4 hours in a thermocycler (MiniCycler™—MJ Research, Watertown, Mass.). Following incubation, 100 μL of 2× OliGreen® reagent (Invitrogen/Molecular Probes, Eugene, Oreg.) in 1×TE was added to each tube, incubated for ˜16 hours at room temperature and then imaged with 4 second exposures. Fluorescence images are provided in FIG. 12.

There was no observable fluorescence from OliGreen® when added exogenously to heat-inactivated control microcapsules, either without exogenously added dNTPs (FIGS. 12G-H) or with exogenously added dNTPs (FIGS. 12I-L). However, microcapsules containing all components necessary to support RCA, either without exogenously added dNTPs (FIGS. 12A-B) or with exogenously added dNTPs (FIGS. 12C-F), demonstrated strong fluorescence from exogenously added OliGreen® providing evidence for significant DNA amplification.

### EXAMPLE 9

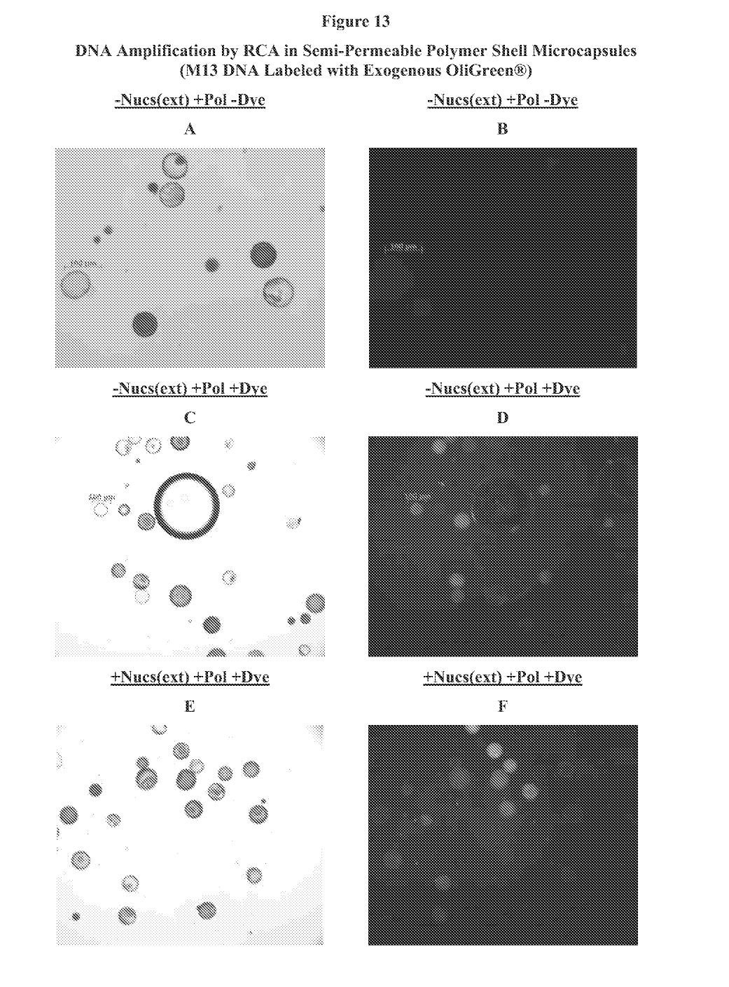

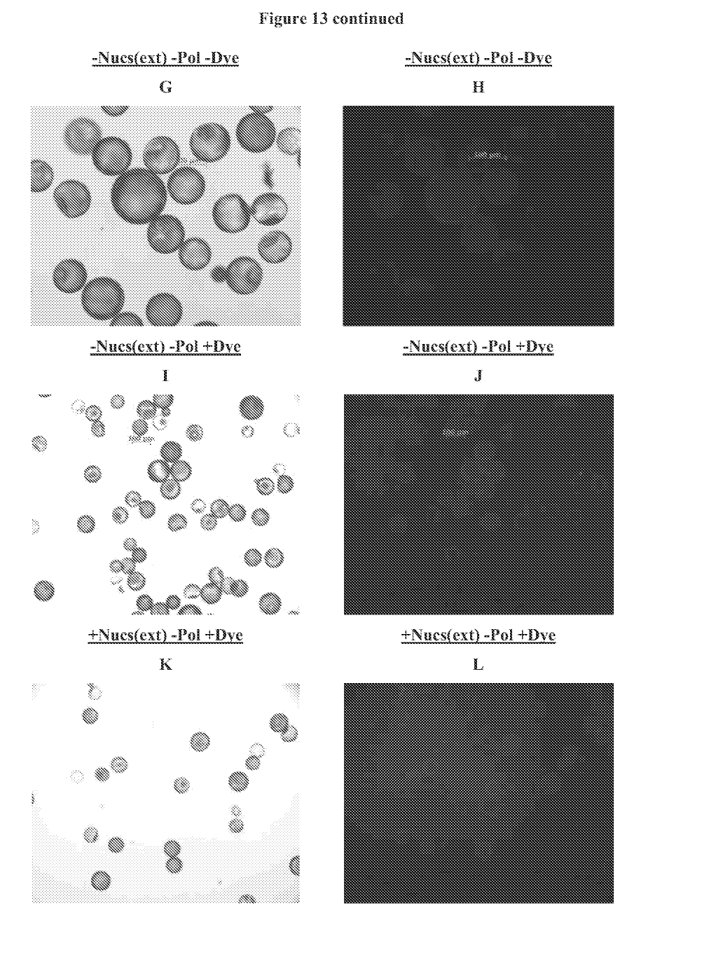

Hyperbranched Rolling Circle Amplification (RCA) was further demonstrated with an alternative polymer shell formulation. Conditions were identical to those in Example 8 except for the composition of the Polymer Shell solution, which was identical to that used in Example 6, except for the ratio of PEGDMA 200 and MPEOEA, which was 10:1 v/v. The microfuge tubes containing the microcapsules were incubated at 35° C. for 10 hours in a thermocycler (MiniCycler™—MJ Research, Watertown, Mass.). Following incubation, 60 μL of 1:20 dilution of OliGreen® reagent (Invitrogen/Molecular Probes, Eugene, Oreg.) in 1× TE was added to each tube, incubated for ˜3 hours at room temperature and then imaged (exposure time=3 sec). Brightfield and fluorescence images are shown in FIG. 13. All control microcapsules lacking polymerase were negative for amplification (G-L). Microcapsules with only internally added nucleotides (C-D), as well as those with both internally and externally added nucleotides (E-F), both show clear evidence of DNA amplification, with somewhat higher integrated fluorescence intensity in the latter batch indicating a higher degree of amplification.

### EXAMPLE 10

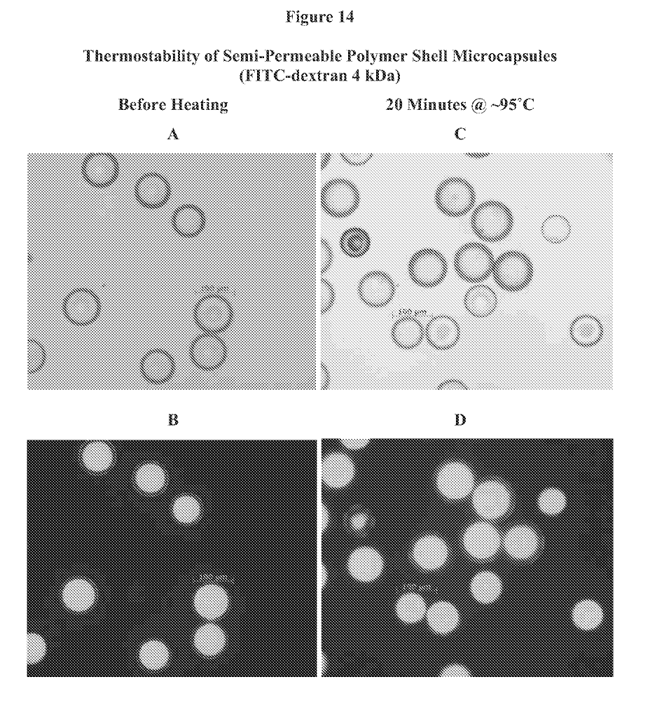

Thermostability of semi-permeable polymer shell microcapsules was demonstrated by producing FITC-labeled dextran (4 kDa) loaded microcapsules as described in Example 5. The harvested microcapsules were rinsed in distilled water and imaged ˜5 minutes after they were created. The microcapsules were then heated to ˜95° C. in distilled water for 20 minutes, cooled to room temperature and reimaged. Brightfield and fluorescence images are provided in FIG. 14.

There was no observable loss of fluorescence or change in the morphology of the microcapsules after heating, indicating that they are sufficiently thermostable to withstand conditions for PCR and/or cycle sequencing.

### EXAMPLE 11

Permeability of the alternative formulation polymer shell microcapsules to dye-labeled dideoxynucleotide terminators was demonstrated using conditions identical to those in Example 7 except for the composition of the Core Solution, which was 25% v/v glycerol. Impermeable microcapsules were used as controls.

5 μL of suspended microcapsules were mixed with 5 μL of dye-labeled dideoxynucleotide terminators (tetramethyl rhodamine-ddTTP, Thermo Sequenase Dye Terminator Cycle Sequencing Core Kit, Amersham Biosciences, Piscataway, N.J.) and incubated at room temperature in the dark for 3 hours.

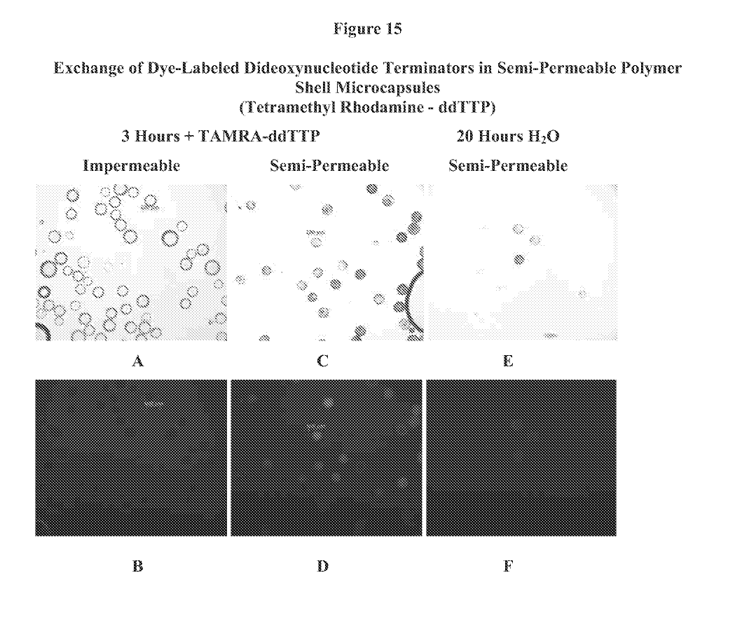

Microcapsules were then washed with 2 mL distilled H₂O and imaged immediately. Microcapsules were allowed to incubate in distilled H₂O for an additional 20 hours in the dark and imaged again as shown in FIG. 15.

The aqueous cores of the impermeable PEGDMA microcapsules were non-fluorescent after 3-hour incubation in dye-labeled ddTTP, whereas the aqueous cores of the semi-permeable PEGDMA-MPEOEA microcapsules show significant internal fluorescence. The process is reversible, as indicated by the loss of internal fluorescence upon further incubation in water, indicating that dye-labeled dideoxynucleotide terminators can freely exchange across the semi-permeable polymer shell membrane of these microcapsules.

Although the present invention has been described in detail with reference to examples above, it is understood that various modifications can be made without departing from the spirit of the invention. Accordingly, the invention is limited only by the following claims. Additional examples and embodiments can be found in co-filed and co-pending patent applications: U.S. patent application Ser. No. 12/221,791, entitled “Microcapsules and methods of use for amplification and sequencing”, and filed on Aug. 6, 2008; U.S. patent application Ser. No. 12/221,794, entitled “Continuous film electrophoresis”, and filed on Aug. 6, 2008; and U.S. patent application Ser. No. 12/221,793, entitled “Continuous imaging of nucleic acids”, and filed on Aug. 6, 2008, each of which is hereby incorporated by reference in its entirety. All cited patents, patent applications and publications referred to in this application are herein incorporated by reference in their entirety.

1. A method of making microcapsules for use in a polymerase-mediated reaction, comprising:
(a) receiving an inner liquid flow, a middle fluid flow and an outer fluid flow into a multiple sheath flow device through separate inlet connections, wherein said inner liquid flow comprises one or more nucleic acid templates and one or more polymerases, said middle fluid flow comprises a semipermeable membrane forming material, and said outer fluid flow comprises a solution or gas; and
(b)focusing said inner liquid flow and said middle fluid flow in at least one aperture of said multiple sheath flow device.

2. The method of claim 1, wherein said outer fluid flow entrains said innermost fluid flow and said middle fluid flow in said aperture.

3. The method of claim 1, further wherein said innermost liquid flow and said semipermeable membrane forming material exit said multiple sheath flow device through said at least one aperture as a liquid jet.

4. The method of claim 3, further wherein breakup of said liquid jet results in formation of microcapsules.

5. The method of claim 4, wherein said microcapsules are monodisperse.

6. The method of claim 1, wherein said at least one aperture is a precision sapphire aperture.

7. The method of claim 1, wherein said semipermeable membrane forming material is polymerized upon exposure to said outer fluid flow of solution or gas.

8. The method of claim 1, wherein said semipermeable membrane forming material is polymerized upon exposure to light.

9. The method of claim 1, wherein said inner fluid flow enters said multiple sheath flow device through an inner fluid inlet connection, said middle fluid flow enters said multiple sheath flow device through a middle fluid inlet connection and said outer fluid flow enters said multiple sheath flow device through a focusing fluid inlet connection inlet connection.

10. The method of claim 9, wherein said inner fluid inlet connection, said middle fluid inlet connection and said outer fluid inlet connection are inert capillary tubing connections.

11. The method of claim 1, wherein said microcapsules are 1 to 10 μm in diameter.

12. The method of claim 11, wherein said microcapsules comprise a diameter with a coefficient of variation that is less than or equal to 10%.

13. The method of claim 1, wherein said multiple sheath flow device is computer operated.

14. The method of claim 13, wherein said computer controls syringe pumps which inject said innermost liquid flow, said semipermeable membrane forming material and said outer fluid flow of solution or gas into said multiple sheath flow device.

15. The method of claim 14, wherein said microcapsules are formed at a rate of at least about 50,000 microcapsules per second, at least about 100,000 microcapsules per second or at least about 500,000 microcapsules per second or at least about 1,000,000 microcapsules per second.

16. The method of claim 1, wherein the polymerase is selected from DNA polymerase or RNA polymerase.

17. The method of claim 16, wherein the DNA polymerase is selected from the group consisting of Taq DNA polymerase I, DNA polymerase II, DNA polymerase III holenzyme, DNA polymerase IV, terminal transferase, Klenow fragment, T4 DNA polymerase, T7 DNA polymerase, phi29 DNA polymerase, and “hot start” polymerases.

18. A method of making microcapsules for use in a enzyme-mediated reaction, comprising:
(a) receiving an inner liquid flow, a middle fluid flow and an outer fluid flow into a multiple sheath flow device through separate inlet connections, wherein said inner liquid flow comprises a nucleic acid template and one or more enzymes, said middle fluid flow comprises a semipermeable membrane forming material, and said outer fluid flow comprises a solution or gas; and
(b) focusing said inner liquid flow with said middle fluid flow in an aperture of said multiple sheath flow device.

19. The microcapsule of claim 18, wherein the enzyme is selected from the group consisting of polymerases, reverse transcriptases, ligases, Klenow fragment and restriction endonucleases, thermophilic DNA polymerases, and “hot start” polymerases.

20. The microcapsule of claim 19, wherein the enzymes are RNA polymerases.
